(19) United States
(12) Patent Application Publication
(10) Pub. No.: US 2014/0051858 A1
(43) Pub. Date: Feb. 20, 2014

(54) PREPARING METHOD OF SOLID CARBAMIC ACID DERIVATIVES

(71) Applicant: Sogang University Research Foundation, Seoul (KR)

(72) Inventors: Nam Hwi HUR, Seoul (KR); Byeong No LEE, Yongin-si (KR)

(73) Assignee: Sogang University Research Foundation, Seoul (KR)

(21) Appl. No.: 13/967,036

(22) Filed: Aug. 14, 2013

Related U.S. Application Data
(63) Continuation of application No. PCT/KR2012/001046, filed on Feb. 13, 2012.

(30) Foreign Application Priority Data
Feb. 14, 2011 (KR) 10-2011-0012972

(51) Int. Cl. C07D 295/13; C07C 209/46; C07C 269/04

(52) U.S. Cl. CPC C07D 295/13; C07C 269/04; C07C 209/46
USPC 544/400; 562/550; 562/443; 564/468; 564/373; 544/402

(57) ABSTRACT

The present disclosure relates to a preparation method for powder of a carbamic acid derivative, which includes reacting a liquid amine derivative with carbon dioxide at a temperature in a range of from about −30° C. to about 500° C. at a pressure in a range of from about 0.3 MPa to about 100 MPa. In addition, the present disclosure relates to a reduction method for powder of a carbamic acid derivative to a liquid amine derivative and carbon dioxide, which includes dissolving powder of the carbamic acid derivative prepared in a solvent; refluxing the carbamic acid derivative at a temperature in a range of from about 30° C. to about 100° C.; and evaporating the solvent. The preparation method for a carbamic acid derivative powder according to the present disclosure enables easy conversion into pure powder of solid carbamic acid derivative without by-products and can remarkably reduce time and energy required for solidification by reacting carbon dioxides and amines with carbon dioxides in high pressure conditions without the use of a solvent. In addition, the prepared solid compounds can be used as a liquid amine substitute or used in a carbamic acid derivative form as necessary.

### TECHNICAL FIELD

The present disclosure relates to a preparation method for a solid carbamic acid derivative and a reduction method for the solid carbamic acid derivative, and more specifically, to a conversion method for a liquid amine derivative to a solid carbamic acid derivative through a reaction with carbon dioxide and a reduction method for the solid carbamic acid derivative to the liquid amine derivative and the carbon dioxide.

### BACKGROUND ART

An amine compound refers to a compound, such as an ammonia molecule, containing a nitrogen atom having an unshared electron pair and is typically basic. Such an ammine compound has been found from both plants and animals. Amine compounds extracted from plants include nicotine contained in tobacco and cocaine which can cause hallucination. Many of drugs daily used contain an amine functional group. A typical drug is penicillin. Further, dopamine well known as a material that stimulates brain nerves and phenylethylamine as a main substance of chocolate are typical compounds each containing an amine functional group. In many cases, amine compounds each having a relatively small molecular weight stay in liquid form at room temperature under atmospheric pressure, give out smell of fish, and cause skin allergies. Further, such liquid amines easily react with oxygen or the like in the air so as to be changed, and, thus, if they are not completely sealed, it is difficult to maintain purity thereof. Therefore, liquid amines are difficult to handle and store and are limited in applications thereof.

As a method for solving the above problem, a solid amine salt prepared by reacting amine with an acid has been used in substitution for amine. Such an amine salt is prepared as a solid salt by adding a strong acid such as a sulfuric acid or a hydrochloric acid to liquid amine with a precipitation reaction. By way of example, when a hydrochloric acid solution is put into the phenylethylamine, a precipitation reaction occurs in an instant with conversion into a phenylethylamine hydrochloric acid which has been actually used in substitution for liquid amine. Such solid amine salts stay in stable solid form at room temperature and have characteristics similar to those of liquid amine when they are dispersed in a solution and react with other compounds. Further, such salts have a very high solubility to water and are useful for utilizing amine. In particular, a drug containing an amine functional group which is not dissolved well in water due to its high molecular weight is used as being bonded to an acid such as a hydrochloric acid. This is because if the drug is converted into a salt, a solubility is sharply increased in an aqueous solution and thus the drug is suitable for use as a drug.

However, such amine salts need a solvent when being generated, the solvent needs to be removed after a reaction due to unnecessary materials added to generate the salts, and an additional process for removing a remaining material is needed. Further, a neutralization process for removing a strong acid such as a hydrochloric acid or a sulfuric acid used in a process for preparing an amine salt is essential. Such a multistep process causes environmental pollution.

As described above, as one of alternatives to solve such problems of the liquid amine and the solid amine salt, there is a method for preparing solid amine derivatives from liquid amine by using eco-friendly carbon dioxide as a reactant. If a reaction is made between liquid amine and carbon dioxide, the carbon dioxide is generally inserted into N—H bonds of amine molecules and converted into carbamic acid derivatives. Most of these carbamic acid derivatives stay in stable solid form in the air. Further, they can be easily separated into the amine and the carbon dioxide by a slight external change, and, thus, they can act as a good alternative to solve the problems of the liquid amine and the solid amine salt.

Many methods for preparing new compounds using carbon dioxide as a reactant have been known so far, and various methods for preparing new compounds by the methods have been reported [Sakakura, T.; Choi, J.-C.; Yasyda, H., Chem. Rev., 2007, 107, 2365]. Further, it is known that carbamic acid derivatives can be formed by a reaction between amine and carbon dioxide. However, it is known that in most of the conventionally known reactions between amine and carbon dioxide, while a carbon dioxide gas is discharged under atmospheric pressure for a long time by using a solvent, a compound is formed in a reactor and used, without an additional separation process, for an intermediate step in a process for forming another compound, or a compound is synthesized in gel form (U.S. Pat. No. 3,551,226), or a compound is formed by using a polar solvent with an ionic liquid [1) Jessop, P. G.; Heldebrant, D. J.; Li, X.; Eckert, C. A.; Liotta, C. L., Nature, 2005, 436, 1102, 2) Lam Phan; Andreatta J. R.; Horvey, L. K.; Edie, C. F.; Luco. Aimeé-L.; Mirchandani, A.; Darensbourg, D. J.; Jessop, P. G., J. Org. Chem., 2008, 73, 127-132, 3) Liu, Y.; Tang, Y.; Barashkov, N. N.; Irgibaeva, I. S.; Y. Lam, J.; Hu, W. R.; Birimzhanova, D.; Yu, Y.; Tang, B. Z., J. Am. Chem. Soc., 2010, 132, 13951.].

In U.S. Pat. No. 3,551,226, it is described that liquid amines react with carbon dioxide under atmospheric pressure at a low temperature for a long time and are converted into and used as a gel type compound. Such a gel type compound contains amine and carbon dioxide at an irregular ratio and is a very sticky liquid, and, thus, it is difficult to separate the gel type compound into pure solid amine in powder form by a typical drying method. Further, if amine is solidified by using carbon dioxide under atmospheric pressure, a sticky gel type compound is obtained rather than solid compound powder. As described above, it is known that carbamic acid derivatives can be formed by a reaction between amine and carbon dioxide. However, there is no research result regarding a separation and preparation method for a solid carbamic acid derivative in powder form.

### DISCLOSURE OF THE INVENTION

### Problems to be Solved by the Invention

In order to solve problems of an amine derivative in liquid form at room temperature under atmospheric pressure, an objective of the present disclosure is to provide a preparation method for powder of a solid carbamic acid derivative from a liquid amine derivative by using carbon dioxide as a reactant.

Further, another objective of the present disclosure is to provide a reduction method for powder of the solid carbamic acid derivative to the liquid amine derivative and the carbon dioxide.

### Means for Solving the Problems

In accordance with an aspect of the present disclosure, there is provided a preparation method for powder of a carbamic acid derivative, including: reacting a liquid amine derivative with carbon dioxide at a temperature in a range of from about −30° C. to about 500° C. at a pressure in a range of from about 0.3 MPa to about 100 MPa.

In accordance with another aspect of the present disclosure, there is provided a reduction method for powder of a carbamic acid derivative to a liquid amine derivative and carbon dioxide, including: dissolving powder of the carbamic acid derivative prepared by the preparation method of the present disclosure in a solvent; refluxing the carbamic acid derivative at a temperature in a range of from about 30° C. to about 100° C.; and evaporating the solvent.

### Effect of the Invention

According to the present disclosure, in a preparation method for powder of a carbamic acid derivative, amine and carbon dioxide react with each other at a high pressure without using a solvent, and, thus, they can be easily converted into pure powder of a solid carbamic acid derivative without by-products and time and energy required for solidification can be remarkably reduced. In the method of the present disclosure, an undiluted solution of liquid amine can be used without using a solvent, and, thus, a very high productivity can be obtained in a relatively small reactor as compared with a case where a solvent is used. Further, remaining impurities contained in a liquid amine derivative can be minimized, and, thus, very pure powder of a carbamic acid derivative with few impurities can be prepared.

In particular, the powder of the carbamic acid derivative prepared according to the present disclosure stay in stable solid state at room temperature as compared with the liquid amine derivative. Thus, (1) amine vapor is not produced, so that the powder of the carbamic acid derivative is odorless; (2) the powder of the carbamic acid derivative is not easily oxidized in the air, so that it is easy and safe to handle and use; (3) the powder of the carbamic acid derivative can be easily separated into the amine and the carbon dioxide during the reaction, so that it has a reactivity equivalent to that of the liquid amine derivative; (4) the powder of the carbamic acid derivative can be used in conditions without a solvent; (5) the powder of the carbamic acid derivative does not contain impurities, so that less by-products are produced through a side reaction; (6) general toxicities of the liquid amine derivative to humans are remarkably reduced; (7) the powder of the carbamic acid derivative is hardly inflammable; and (8) the powder of the carbamic acid derivative contains few impurities, so that it can be reduced to a liquid amine derivative having a very high purity.

### BRIEF DESCRIPTION OF THE DRAWINGS

FIG. 1 provides a photo of powder of carbamic acid derivatives prepared in Example 1 and Examples 4 to 8 of the present disclosure.

### BEST MODE FOR CARRYING OUT THE INVENTION

Hereinafter, illustrative embodiments and examples of the present disclosure will be described in detail with reference to the accompanying drawings so that the present disclosure may be readily implemented by those skilled in the art. However, it is to be noted that the present disclosure is not limited to the illustrative embodiments and examples but can be embodied in various other ways. In drawings, parts irrelevant to the description are omitted for the simplicity of explanation, and like reference numerals denote like parts through the present disclosure.

Through the present disclosure, the term “connected to” or “coupled to” that is used to designate a connection or coupling of one element to another element includes both a case that an element is “directly connected or coupled to” another element and a case that an element is “electronically connected or coupled to” another element via still another element.

Through the present disclosure, the term “on” that is used to designate a position of one element with respect to another element includes both a case that the one element is adjacent to the another element and a case that any other element exists between these two elements.

Through the present disclosure, the term “comprise or include” and/or “comprising or including” used in the document means that one or more other components, steps, operation and/or existence or addition of elements are not excluded in addition to the described components, steps, operation and/or elements unless context dictates otherwise.

Through the present disclosure, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party.

Through the present disclosure, the term “step of” does not mean “step for”.

Through the present disclosure, the term “combinations of” included in Markush type description means mixture or combinations of one or more components, steps, operations and/or elements selected from a group consisting of components, steps, operation and/or elements described in Markush type and thereby means that the disclosure includes one or more components, steps, operations and/or elements selected from the Markush group.

Through the present disclosure, a phrase in the form “A and/or B” means “A or B, or A and B”.

Through the present disclosure, the term “alkyl group” may include linear or branched, saturated or unsaturated C₁₋₃₀ alkyl groups and may include, for example methyl, ethyl, propyl, butyl, pentyl, hexyl, hepxyl, octyl, nonyl, decyl, undecyl, dodecyl, tridecyl, tetradecyl, pentadecyl, hexadecyl, heptadecyl, octadecyl, nonadecyl, acosanyl, or all available isomers thereof, but the present disclosure may not be limited thereto.

Hereinafter, illustrative embodiments of the present disclosure will be explained in detail, but the present disclosure may not be limited thereto.

In accordance with an aspect of the present disclosure, there is provided a preparation method for powder of a carbamic acid derivative, including reacting a liquid amine derivative with carbon dioxide at a temperature in a range of from about −30° C. to about 500° C. at a pressure in a range of from about 0.3 MPa to about 100 MPa.

In accordance with an illustrative embodiment of the present disclosure, the liquid amine derivative may include a compound represented by the following Chemical Formula 1, but may not be limited thereto:

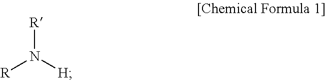

wherein in Chemical Formula 1, R and R′ independently represent hydrogen; a C₁₋₃₀ alkyl group substituted or non-substituted by nitrogen; a phenyl group substituted or non-substituted by nitrogen; a C₆₋₃₀ aryl group substituted or non-substituted by nitrogen; or a C₃₋₃₀ cyclo alkyl group substituted or non-substituted by nitrogen, respectively.

In accordance with an illustrative embodiment of the present disclosure, the prepared powder of the carbamic acid derivative may include a compound represented by the following Chemical Formula 2 or Chemical Formula 3, but may not be limited thereto:

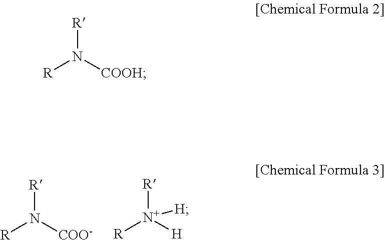

wherein in Chemical Formula 2 and Chemical Formula 3, R and R′ independently represent hydrogen; a C₁₋₃₀ alkyl group substituted or non-substituted by nitrogen; a phenyl group substituted or non-substituted by nitrogen; a C₆₋₃₀ aryl group substituted or non-substituted by nitrogen; or a C₃₋₃₀ cyclo alkyl group substituted or non-substituted by nitrogen, respectively.

In accordance with an illustrative embodiment of the present disclosure, a content of an amine group (—NH) in the amine derivative may be from about 50 wt % to about 99 wt %, but may not be limited thereto.

In accordance with an illustrative embodiment of the present disclosure, the temperature may be in a range of from about −30° C. to about 500° C., but may not be limited thereto. During the reaction, if the temperature is too low or too high, a system or costs may be unnecessarily consumed to maintain a low temperature or a high temperature. By way of example, the temperature may be in a range of from about −30° C. to about 500° C., from about −30° C. to about 400° C., from about −30° C. to about 300° C., from about −30° C. to about 200° C., from about −30° C. to about 100° C., from about −30° C. to about 50° C., from about −30° C. to about 30° C., from about −30° C. to about 20° C., from about −30° C. to about 10° C., from about −30° C. to about 0° C., from about −30° C. to about −10° C., from about −30° C. to about −20° C., from about −20° C. to about 500° C., from about −01° C. or to about 500° C., from about 0° C. to about 500° C., from about 10° C. or to about 500° C., from about 20° C. to about 500° C., from about 30° C. to about 500° C., from about 50° C. to about 500° C., from about 100° C. to about 500° C., from about 200° C. to about 500° C., from about 300° C. to about 500° C., from about 400° C. to about 500° C., or from about 0° C. to about 300° C., but the present disclosure may not be limited thereto.

In accordance with an illustrative embodiment of the present disclosure, the pressure may be in a range of from about 0.3 MPa to about 100 MPa, but may not be limited thereto. During the reaction, if the pressure is less than about 0.3 MPa, powder of a carbamic acid derivative may not be formed and a gel type compound may be formed. By way of example, the pressure may be in a range of from about 0.3 MPa to about 100 MPa, from about 0.3 MPa to about 90 MPa, from about 0.3 MPa to about 80 MPa, from about 0.3 MPa to about 70 MPa, from about 0.3 MPa to about 60 MPa, from about 0.3 MPa to about 50 MPa, from about 0.3 MPa to about 40 MPa, from about 0.3 MPa to about 30 MPa, from about 0.3 MPa to about 20 MPa, from about 0.3 MPa to about 10 MPa, from about 0.3 MPa to about 5 MPa, from about 0.3 MPa to about 1 MPa, from about 1 MPa to about 100 MPa, from about 5 MPa to about 100 MPa, from about 10 MPa to about 100 MPa, from about 20 MPa to about 100 MPa, from about 30 MPa to about 100 MPa, from about 40 MPa to about 100 MPa, from about 50 MPa to about 100 MPa, from about 60 MPa to about 100 MPa, from about 70 MPa to about 100 MPa, from about 80 MPa to about 100 MPa, from about 90 MPa to about 100 MPa, or from about 1 MPa to about 50 MPa, but the present disclosure may not be limited thereto.

The reaction may be made by adding the carbon dioxide to the liquid amine derivative, and even when the carbon dioxide is added to an additional solvent besides the liquid amine derivative, pure powder of a solid carbamic acid derivative can be prepared in an equivalent manner to a case where the liquid amine derivative reacts with the carbon dioxide without a solvent.

In accordance with an illustrative embodiment of the present disclosure, the preparation method for powder of the carbamic acid derivative of the present disclosure may include reacting a mixed solution of the liquid amine derivative with ethers, alcohols, aliphatic hydrocarbons, carbocycles, heterocycles, aromatics, substituted heteroaromatic cycles, with the carbon dioxide, but may not be limited thereto. As described above, if an additional solvent is input besides the liquid amine derivative, a content of the amine derivative in the mixed solution may be from about 1 wt % to about 99 wt %, for example from about 1 wt % to about 99 wt %, from about 1 wt % to about 90 wt %, from about 1 wt % to about 80 wt %, from about 1 wt % to about 70 wt %, from about 1 wt % to about 60 wt %, from about 1 wt % to about 50 wt %, from about 1 wt % to about 40 wt %, from about 1 wt % to about 30 wt %, from about 1 wt % to about 20 wt %, from about 1 wt % to about 10 wt %, from about 1 wt % to about 5 wt %, from about 5 wt % to about 99 wt %, from about 10 wt % to about 99 wt %, from about 20 wt % to about 99 wt %, from about 30 wt % to about 99 wt %, from about 40 wt % to about 99 wt %, from about 50 wt % to about 99 wt %, from about 60 wt % to about 99 wt %, from about 70 wt % to about 99 wt %, from about 80 wt % to about 99 wt %, or from about 90 wt % to about 99 wt %, but the present disclosure may not be limited thereto.

In accordance with an illustrative embodiment of the present disclosure, the preparation method for powder of the carbamic acid derivative, the method may further include after the reaction with the carbon dioxide, reducing the pressure to be in a range of from about 0.01 MPa to about 0.1 MPa so as to evaporate excess carbon dioxide, but the present disclosure may not be limited thereto.

In accordance with an illustrative embodiment of the present disclosure, the preparation method for powder of the carbamic acid derivative, the method may further include washing the prepared powder of the carbamic acid derivative with C₁-C₁₂ alcohols, tetrahydrofuran, ethers, dimethylformamide, or mixed solutions thereof, and drying the powder of the carbamic acid derivative, but the present disclosure may not be limited thereto. If the method further includes the washing and drying step, impurities in the liquid amine derivative are removed and powder of the carbamic acid derivative having a higher purity can be prepared.

In accordance with an illustrative embodiment of the present disclosure, the carbon dioxide may include gas-phase carbon dioxide, liquid-phase carbon dioxide, carbon dioxide in a supercritical state, or solid-phase dry ice, but the present disclosure may not be limited thereto.

In accordance with another aspect of the present disclosure, there is provided a reduction method for powder of the carbamic acid derivative to a liquid amine derivative and carbon dioxide, the method including: dissolving powder of the carbamic acid derivative prepared by the method of the above-described aspect of the present disclosure in a solvent; refluxing the carbamic acid derivative in the solvent at a temperature in a range of from about 30° C. to about 100° C.; and evaporating the solvent.

In accordance with an illustrative embodiment of the present disclosure, the solvent is not particularly limited as long as it can dissolve a solid carbamic acid derivative and can be easily separated from the prepared liquid amine derivative and may include, for example, C₁-C₁₂ alcohols, C_{2-C}₁₂ ethers, and the like, but may not be limited thereto.

In accordance with an illustrative embodiment of the present disclosure, the temperature may be in a range of from about 30° C. to about 100° C., from about 30° C. to about 90° C., from about 30° C. to about 80° C., from about 30° C. to about 70° C., from about 30° C. to about 60° C., from about 30° C. to about 50° C., from about 30° C. to about 40° C., from about 40° C. to about 100° C., from about 50° C. to about 100° C., from about 60° C. to about 100° C., from about 70° C. to about 100° C., from about 80° C. to about 100° C., from about 90° C. to about 100° C., or from about 50° C. to about 80° C., but the present disclosure may not be limited thereto.

### MODE FOR CARRYING OUT THE INVENTION

Hereinafter, the present invention will be explained in detail with reference to examples. However, it is clear that the following examples are provided to illustrate the present invention but not to limit the present invention.

### EXAMPLE 1

3 g of ehtylenediamine (H₂NCH₂CH₂NH₂) was put into a pressure vessel (Parr 4714) with a volume of 45 mL without a solvent and reacted at a carbon dioxide pressure of 3 MPa at a temperature of 50° C. for 5 hours so as to obtain a solid ehtylenediamine derivative. By putting the pressure vessel in an oil bath, the temperature was adjusted. After the reaction, remaining carbon dioxide was removed, and a remaining solid was washed with 20 mL of ethanol and 20 mL of diethylether five times and dried in a vacuum for 3 hours so as to obtain powder of a solid carbamic acid derivative.

In order to confirm characteristics of the obtained solid powder, elementary analysis and nuclear magnetic resonance (NMR) spectroscopy analysis were carried out. According to results of the elementary analysis and the nuclear magnetic resonance spectroscopy analysis, it was confirmed that the obtained solid powder was a 2-aminoethyl carbamic acid compound represented by a chemical formula of H₂NCH₂CH₂NHCOOH. A yield of the obtained H₂NCH₂CH₂NHCOOH was 98% or more based on the used ehtylenediamine.

The results of the elementary analysis (unit: %) and the ¹H NMR analysis on the H₂NCH₂CH₂NHCOOH as a product were as follows:

Elementary analysis (calculated value, experimental value): C (34.61, 34.68), H (7.75, 7.71), N (26.91, 26.93);

¹H NMR (400 MHz, CD₃OD, 27° C.) δ=4.659 (s, 3H, —NH₃), 3.005 (t, 2H, —CH₂NHCOO—), 2.765 (t, 2H, —CH₂NH₃), 2.681 (s, 1H, —NH).

### COMPARATIVE EXAMPLE 1

Comparative Example 1 was carried out in the same manner as Example 1 except that 3 g (50.0 mmol) of ehtylenediamine was used and carbon dioxide was bubbled under atmospheric pressure at a temperature of 0° C. for 16 hours.

In this case, the ehtylenediamine was converted into transparent gel type compound having a very high viscosity, but solids were not produced. This result is the same as described in U.S. Pat. No. 3,551,226, and it is determined that not a single product but a mixed product was synthesized.

### EXAMPLE 2

Example 2 was carried out in the same manner as

Example 1 except that diethylether (2 mL) was used as a solvent and 2 g (33.3 mmol) of ehtylenediamine was used so as to obtain powder of a solid carbamic acid derivative. In order to confirm characteristics of the obtained powder of the carbamic acid derivative, an elementary analysis and a nuclear magnetic resonance spectroscopy analysis were carried out in the same manner as Example 1.

A result of the elementary analysis (unit: %) on the obtained solid powder was almost the same as that of Example 1, and a result of the ¹H NMR analysis was exactly the same as that of Example 1. A yield of a product was 98% or more based on the used ehtylenediamine. According to the results of the elementary analysis and the ¹H NMR analysis, it was confirmed that the powder prepared in Example 2 was H₂NCH₂CH₂NHCOOH as the same material as that of Example 1.

### EXAMPLE 3

Example 3 was carried out in the same manner as Example 1 except that 10 g of solid-phase carbon dioxide (dry ice) was used as a source of carbon dioxide instead of gas-phase carbon dioxide and reacted with ehtylenediamine so as to obtain powder of a solid carbamic acid derivative. In order to confirm characteristics of the obtained powder of the carbamic acid derivative, an elementary analysis and a nuclear magnetic resonance spectroscopy analysis were carried out in the same manner as Example 1.

According to the results of the elementary analysis and the ¹H NMR analysis on the obtained solid powder, it was confirmed that the powder prepared in Example 3 was H₂NCH₂CH₂NHCOOH as the same material as that of Example 1.

### EXAMPLE 4

Example 4 was carried out in the same manner as Example 1 except that at a temperature of about 25° C., 3 g (52.5 mmol) of allylamine (CH₂═CHCH₂NH₂) was used as a reactant instead of ehtylenediamine so as to obtain solid powder of a carbamic acid derivative. In order to confirm characteristics of the obtained powder of the carbamic acid derivative, an elementary analysis was carried out in the same manner as Example 1.

According to the results of the elementary analysis and a mass spectrometric analysis, it was confirmed that the obtained solid powder was prop-2-en-1-aminium allylcarbamate ((CH₂═CHCH₂NH₂)₂CO₂). A yield of the obtained solid powder was 96% or more based on the used allylamine.

The result of the elementary analysis (unit: %) was as follows:

Elementary analysis (calculated value, experimental value): C (53.14, 53. 09), H (8.92, 8.97), N (17.71, 17.65).

### EXAMPLE 5

Example 5 was carried out in the same manner as Example 1 except that 3 g (28.0 mmol) of benzylamine (C₆H₅CH₂NH₂) was used as a reactant instead of ehtylenediamine so as to obtain powder of a solid carbamic acid derivative. In order to confirm characteristics of the obtained powder of the carbamic acid derivative, an elementary analysis was carried out in the same manner as Example 1.

According to the results of the elementary analysis and a mass spectrometric analysis, it was confirmed that the obtained solid powder was phenylmethanaminium benzylcarbamate ((C₆H₅CH₂NH₂)₂CO₂). A yield of the obtained solid powder was 98% or more based on the used benzylamine.

The result of the elementary analysis (unit: %) was as follows:

Elementary analysis (calculated value, experimental value): C (69.74, 69.91), H (7.02, 7.18), N (10.85, 10.82).

### EXAMPLE 6

Example 6 was carried out in the same manner as Example 1 except that 3 g (15.2 mmol) of benzylamine [(C₆H₅CH₂)₂NH] was used as a reactant instead of ehtylenediamine so as to obtain powder of a solid carbamic acid derivative. In order to confirm characteristics of the obtained powder of the carbamic acid derivative, an elementary analysis was carried out in the same manner as Example 1.

According to the results of the elementary analysis and a mass spectrometric analysis, it was confirmed that the obtained solid powder was dibenzylammonium dibenzylcarbamate [({(6H₅CH₂)₂NH₂}₂CO₂] A yield of the obtained solid powder was 98% or more based on the used dibenzylamine.

The result of the elementary analysis (unit: %) was as follows:

Elementary analysis (calculated value, experimental value): C (79.42, 79.45), H (6.90, 7.08), N (6.39, 6.43).

### EXAMPLE 7

Example 7 was carried out in the same manner as Example 1 except that 3 g (14.9 mmol) of 1,4-(bis-aminopropyl)piperazine was used as a reactant instead of ehtylenediamine so as to obtain powder of a solid carbamic acid derivative. In order to confirm characteristics of the obtained powder of the carbamic acid derivative, an elementary analysis was carried out in the same manner as Example 1.

According to the results of the elementary analysis and a mass spectrometric analysis, it was confirmed that the obtained solid powder was 3-(4-(4-(carboxyamino)butyl)piperazin-1-yl)propan-1-aminium. A yield of the obtained solid powder was 98% or more based on the used amine.

The result of the elementary analysis (unit: %) was as follows:

Elementary analysis (calculated value, experimental value): C (54.07, 54.37), H (9.90, 10.10), N (22.94, 23.18).

### EXAMPLE 8

Example 8 was carried out in the same manner as Example 1 except that 3 g (24.7 mmol) of phenylethylamine (C₆H₅CH₂CH₂NH₂) was used as a reactant instead of ehtylenediamine so as to obtain powder of a solid carbamic acid derivative. In order to confirm characteristics of the obtained powder of the carbamic acid derivative, an elementary analysis was carried out in the same manner as Example 1.

According to the results of the elementary analysis and a mass spectrometric analysis, it was confirmed that the obtained solid powder was 2-phenylethanaminium phenethylcarbamate. A yield of the obtained solid powder was 98% or more based on the used amine.

The result of the elementary analysis (unit: %) was as follows:

Elementary analysis (calculated value, experimental value): C (71.30, 71.21), H (7.74, 7.82), N (11.17, 10.98).

### EXAMPLE 9

Example 9 was carried out in the same manner as Example 1 except that 3 g (41.0 mmol) of diethylamine [(CH₃CH₂)₂NH] was used as a reactant instead of ehtylenediamine so as to obtain powder of a solid carbamic acid derivative. In order to confirm characteristics of the obtained powder of the carbamic acid derivative, an elementary analysis was carried out in the same manner as Example 1.

According to the results of the elementary analysis and a mass spectrometric analysis, it was confirmed that the obtained solid powder was diethylammonium diethylcarbamate. A yield of the obtained solid powder was 93% or more based on the used amine.

The result of the elementary analysis (unit: %) was as follows:

Elementary analysis (calculated value, experimental value): C (56.80, 57.01), H (11.65, 11.81), N (16.82, 17.01).

A structure, a reaction time, and a yield of the powder of the solid carbamic acid derivatives produced from Example 1 to Example 9 by reacting the liquid amines with the carbon dioxides were as shown in the following Table 1.

TABLE 1

| | | | Tem- | | | |
|---|---|---|---|---|---|---|
| | | | per- | Pres- | | |
| | Liquid amine | Carbamic acid | ature^{a} | sure | Yield^{b} | Re- |
| | derivative | derivative | (° C.) | (MPa) | (%) | marks |
| Exam- ple 1 | 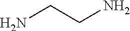 | 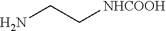 | 50 | 3 | >98 | white pow- der |
| Compar- ative Exam- ple 1^{c} | 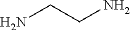 | — | 0 | 0.1 | — | trans- parent gel |
| Exam- ple 2^{d} | 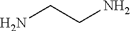 | 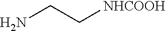 | 50 | 3 | >98 | white pow- der |
| Exam- ple 3^{e} | 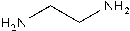 | 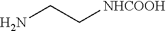 | 50 | 3 | >98 | white pow- der |
| Exam- ple 4 | 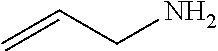 | 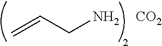 | 25 | 3 | >98 | white pow- der |
| Exam- ple 5 | 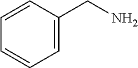 | 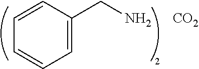 | 50 | 3 | >98 | nee- dle crys- tal |
| Exam- ple 6 | 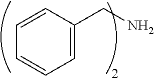 | 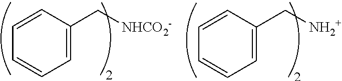 | 50 | 3 | >98 | nee- dle crys- tal |
| Exam- ple 7 | 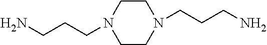 [SMILES: NCCCN1CCN(CCCN)CC1] | 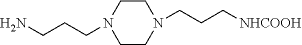 | 50 | 3 | >98 | white crys- tal- line |
| Exam- ple 8 | 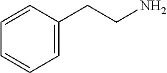 | 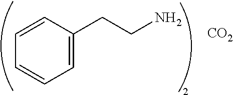 | 50 | 3 | >98 | white crys- tal- line pow- der |
| Exam- ple 9 | 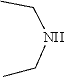 | 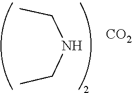 | 50 | 3 | >95 | white pow- der^{f} |

^{a}reaction time: 5 h, P_{CO2} = 3 MPa
^{b}isolation yield, no purification, based on amine
^{c}CO₂: bubbling (1 atm)
^{d}di-ethyl ether: 2 mL
^{e}dry ice: 10 g
^{f}isolated at <10° C., unstable at >10° C.

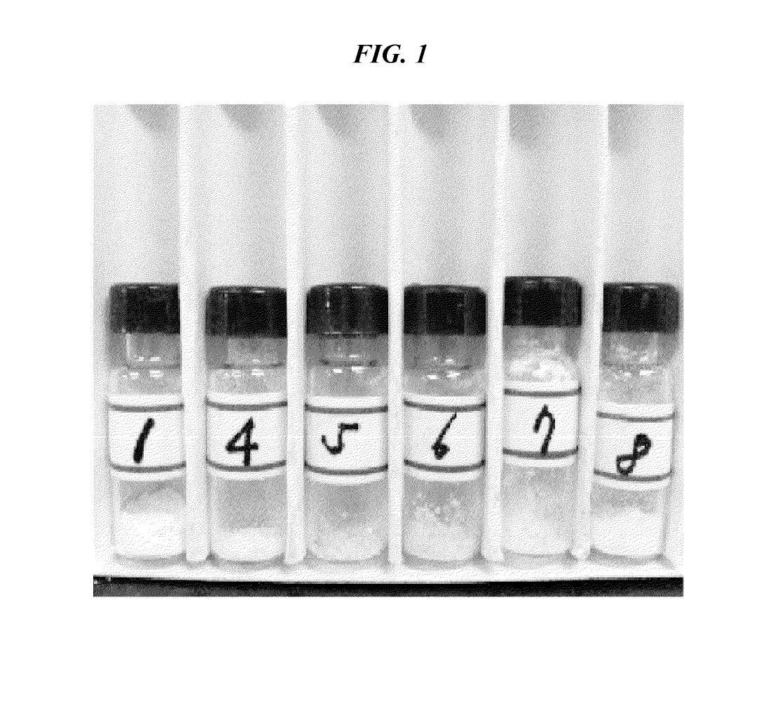

Further, a photo of the solid carbamic acid derivatives prepared in Example 1 and Examples 4 to 8 is shown in FIG. 1. As shown in FIG. 1, it can be confirmed that a carbamic acid derivative in powder form is prepared according to the preparation method for powder of the carbamic acid derivative.

As described above, a solid carbamic acid derivative was synthesized from a liquid amine derivative. The following Examples 10 to 12, and Comparative Example 2 are examples showing that the powder of the carbamic acid derivatives prepared in Example 1 to Example 9 had reactivities equivalent to that of a liquid amine derivative.

### EXAMPLE 10

### Reduction of Powder of the Solid Carbamic Acid Derivative to Liquid Amine Derivative

1 g of 3-(4-(4-(carboxyamino)butyl)piperazin-1-yl)propan-1-aminium as powder of the solid carbamic acid derivative prepared in Example 7 was dissolved in 3 mL of methanol as a solvent and refluxed at a temperature of from 65° C. to 70° C. for 2 hours. Then, a remaining solution was removed by depressurizing a pressure to 0.1 MPa at 0° C. and distilling the methanol so as to obtain a liquid compound. In order to confirm characteristics of the obtained liquid compound, an elementary analysis and a NMR analysis were carried out in the same manner as Example 1.

According to the results of the ¹H NMR analysis and the elementary analysis, it was confirmed that the obtained liquid compound was 1,4-(bis-aminopropyl)piperazine as a liquid amine derivative used as a reactant in Example 7. A yield of the obtained liquid compound was 98% or more based on the used solid compound and a purity of the obtained liquid compound was 99.8% or more.

### EXAMPLE 11

### Reduction of Powder of the Solid Carbamic Acid Derivative to Liquid Amine Derivative

1 g of diethylammonium diethylcarbamate as powder of the solid carbamic acid derivative prepared in Example 9 was dissolved in 3 mL of methanol as a solvent and refluxed at a temperature of from 60° C. to 65° C. for 2 hours. Then, a remaining solution was distilled under reduced pressure to 0.1 MPa at 0° C. so as to obtain a liquid compound. In order to confirm characteristics of the obtained liquid compound, an elementary analysis and a NMR analysis were carried out in the same manner as Example 1.

According to the results of the ¹H NMR analysis and the elementary analysis, it was confirmed that the obtained liquid compound was diethylamine as liquid amine derivative used as a reactant in Example 9. A yield of the obtained liquid compound was 95% or more based on the used solid compound and a purity of the obtained liquid compound was 99.9% or more.

### EXAMPLE 12

### Reaction Between Powder of the Solid Carbamic Acid Derivative and Benzaldehyde

0.791 g (5 mmol) of prop-2-en-1-aminium allylcarbamate [(CH₂═CHCH₂NH₂)₂CO₂] as the solid carbamic acid derivative prepared in Example 4 was dissolved in 30 mL of ether as a solvent and reacted with 1.06 g (10 mmol) of benzaldehyde (C₆H₅CHO) and distilled under reduced pressure to 0.1 MPa so as to obtain a liquid compound. In order to confirm characteristics of the obtained liquid compound, a NMR analysis was carried out in the same manner as Example 1.

According to the result of the ¹H NMR analysis, it was confirmed that the obtained liquid compound was N-benzylideneprop-2-en-1-amine. A yield of the obtained liquid compound was 97% or more based on the used solid compound and a purity of the obtained liquid compound was about 99.5%.

The result of the ¹H NMR analysis was as follows:

¹H NMR (400 MHz, CDCl₃, 27° C. ) δ=8.28 (s, 1H, CH═N), 7.76 (m, 2H, phenyl),7.40 (m, 3H, phenyl), 6.07 (m, 1H, CH═CH₂), 5.23 (dd, 1H, CH₂ₐ=), 5.14 (dd, 1H, CH_{2b}=), 4.25 (dd, 2H, CH₂—N).

### COMPARATIVE EXAMPLE 2

### Reaction Between Powder of the Solid Carbamic Acid Derivative and Benzaldehyde

Comparative Example 2 was carried out in the same manner as Example 12 except that 0.57 g (10 mmol) of allylamine (CH₂═CHCH₂NH₂) was used instead of a solid carbamic acid derivative. After the reaction, a liquid compound was obtained through distillation under reduced pressure to 0.1 MPa. In order to confirm characteristics of the obtained liquid compound, a NMR analysis was carried out in the same manner as Example 1.

The obtained liquid compound was the same as the compound obtained in Example 12. A yield of the obtained liquid compound was 97% or more based on the used allylamine and a purity of the obtained liquid compound was about 99.3%.

The above description of the present disclosure is provided for the purpose of illustration, and it would be understood by those skilled in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

What is claimed is:

1. A preparation method for powder of a carbamic acid derivative, the method comprising:
reacting a liquid amine derivative with carbon dioxide at a temperature in a range of from about −30° C. to about 500° C. at a pressure in a range of from about 0.3 MPa to about 100 MPa.

2. The preparation method of claim 1,
wherein the liquid amine derivative includes a compound represented by the following Chemical Formula 1: 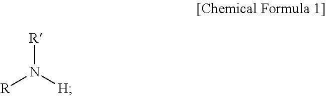
wherein in Chemical Formula 1,
R and R′ independently represent hydrogen; a C₁₋₃₀ alkyl group substituted or non-substituted by nitrogen; a phenyl group substituted or non-substituted by nitrogen; a C₆₋₃₀ aryl group substituted or non-substituted by nitrogen; or a C₃₋₃₀ cyclo alkyl group substituted or non-substituted by nitrogen, respectively.

3. The preparation method of claim 1,
wherein the prepared carbamic acid derivative includes a compound represented by the following Chemical Formula 2 or Chemical Formula 3: 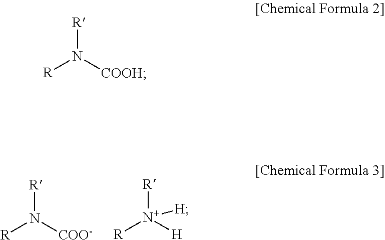
wherein in Chemical Formula 2 and Chemical Formula 3,
R and R′ independently represent hydrogen; a C₁₋₃₀ alkyl group substituted or non-substituted by nitrogen; a phenyl group substituted or non-substituted by nitrogen; a C₆₋₃₀ aryl group substituted or non-substituted by nitrogen; or a C₃₋₃₀ cyclo alkyl group substituted or non-substituted by nitrogen, respectively.

4. The preparation method of claim 1,
wherein a content of an amine group (—NH) in the amine derivative is from about 50 wt % to about 99 wt %.

5. The preparation method of claim 1,
wherein the temperature is in a range of from about 0° C. to about 300° C.

6. The preparation method of claim 1,
wherein the pressure is in a range of from about 1 MPa to about 50 MPa.

7. The preparation method of claim 1, the method including:
reacting a mixed solution of the liquid amine derivative with ethers, alcohols, aliphatic hydrocarbons, carbocycles, heterocycles, aromatics, or substituted heteroaromatic cycles, with the carbon dioxide.

8. The preparation method of claim 7,
wherein a content of the amine derivative in the mixed solution is from about 1 wt % to about 99 wt %.

9. The preparation method of claim 1, further comprising:
after the reaction with the carbon dioxide, reducing the pressure to be in a range of from about 0.01 MPa to about 0.1 MPa so as to evaporate excess carbon dioxide.

10. The preparation method of claim 1, further comprising:
washing the prepared powder of the carbamic acid derivative with C₁-C₁₂ alcohols, tetrahydrofuran, ethers, dimethylformamide, or mixed solutions thereof, and drying the powder of the carbamic acid derivative.

11. The preparation method of claim 1,
wherein the carbon dioxide includes gas-phase carbon dioxide, liquid-phase carbon dioxide, carbon dioxide in a supercritical state, or solid-phase dry ice.

12. A reduction method for powder of a carbamic acid derivative to a liquid amine derivative and carbon dioxide, the method comprising:
dissolving powder of the carbamic acid derivative prepared by the preparation method of any one of claims 1 to 11 in a solvent;
refluxing the carbamic acid derivative at a temperature in a range of from about 30° C. to about 100° C.; and
evaporating the solvent.

13. The reduction method of claim 12,
wherein the solvent includes C₁-C₁₂ alcohols or C₂-C₁₂ ethers.

14. The reduction method of claim 12,
wherein the temperature is in a range of from about 50° C. to about 80° C.
